(19) United States
(12) Patent Application Publication
(10) Pub. No.: US 2025/0009673 A1
(43) Pub. Date: Jan. 9, 2025

(54) ALBUMIN PROTEIN FOR USE AS AN EMULSIFIER AND DRUG CARRIER

(71) Applicant: ECS BRANDS, LTD., Broomfield, CO (US)

(72) Inventors: Arthur JAFFEE, Broomfield, CO (US); Dan SCHULZ, Brooklyn Park (AU)

(21) Appl. No.: 18/705,404

(22) PCT Filed: Oct. 26, 2022
(86) PCT No.: PCT/US2022/047934

§ 371 Date: Apr. 26, 2024

Related U.S. Application Data
(60) Provisional application No. 63/272,088, filed on Oct. 26, 2021.

(51) Int. Cl. A61K 9/50; A61K 9/00; A61K 9/51; A61K 31/00

(52) U.S. Cl. CPC A61K 9/5052; A61K 9/0053; A61K 9/5089; A61K 9/5169; A61K 9/5192; A61K 31/658

(57) ABSTRACT

The present disclosure is directed to methods of using albumin as an emulsifier to assist in blending otherwise immiscible components. Methods use albumin as an emulsifier and a carrier. The disclosure also teaches utilization of albumin as a microencapsulating liquid, gel or powder and as a drug or nutraceutical carrier wherein bioavailability is increased.

### FIELD OF THE INVENTION

The present disclosure relates to the use of albumin as an emulsifier, foaming agent or drug carrier.

### BACKGROUND

Albumin is a protein found in the seeds of some plants e.g., hemp seeds (Cannabis sativa), kidney beans (Phaseolus vulgaris) and locust beans (Parkia biglobosa). Albumin also is a bioactive protein produced in the human body that makes up 55% of the protein in blood plasma. There, it has a variety of functions including blood pressure maintenance and transport of nutrients and other bioactive molecules such as Cannabinoids.

The bioavailability of drugs reaching their intended biological destination is a recognized problem in the medical field. In general, the dose of a drug is indirectly proportional to its bioavailability. Utilization of a carrier protein such as albumin has been shown to help with more efficient delivery of drugs and other compounds that have low bioavailability, as well as to increase the solubility of drugs and other compounds that are poorly soluble in water.

These bioactive molecules are important for regulating blood pressure and are injected into patients when emergency blood pressure regulation as needed such as with burns, shock and liver disease.

As a drug carrier (e.g. for Cannabinoids) albumin may be injected into the human body or consumed orally as a means of raising blood levels of that drug. In human blood, many compounds such as hormones, drugs, fatty acids and steroids are carried by albumin proteins. Due to albumin's short half-life of 20 days in the human body, the body can become deficient in albumin. This deficiency can become life threatening.

Proteins from plant seeds are an abundant alternative to animal-based sources of proteins. In microencapsulation, these proteins are used as a wall forming material for a variety of active compounds. In most cases, two techniques of microencapsulation, spray-drying and coacervation, are used for the preparation of microparticles from plant proteins. Proteins extracted from soy bean, pea and wheat have already been studied as carrier materials for microparticles.

Micro- and nano-encapsulation is a technology used to isolate or deliver liquids or other ingredients to a patient in need. The encapsulated substances are referred to as the core or internal phase and the outer materials are considered the external or coating phase.

Hemp seeds are used worldwide as a source of food and as a nutritional supplement. Hemp inflorescence is rich in non-psychoactive, biologically active cannabinoids. Hemp seed has a pleasant nutty taste and represents a valuable source of essential fatty acids, minerals, vitamins, and fibers, as well as essential amino acids.

Purified hemp protein has traditionally been a low quality, undesirable product due to the type of manufacturing used to produce it. A few negative attributes include course and gritty texture, insolubility in water, dark and “earthy” appearance, and “earthy” flavor.

In the food industry, there is great variability in the emulsifying properties reported for different proteins. While meat proteins tend to be good emulsifying agents, the use of plant proteins as emulsifiers could be advantageous for marketing purposes. In addition to a drug carrier, Albumin can also serve as both an emulsifier and foaming agent.

As outlined above, albumin is a multifaceted, highly soluble, stable, non toxic, non poisonous, biocompatible and biodegradable protein. Because of its versatile nature, it can be used for the delivery of drugs, hormones, metals and fatty acids by binding of these molecules to specific binding sites of albumin. The structure, location, size, charge and hydrophobicity of these drug binding sites are very important to optimize the interaction of drugs with albumin.

Until recently, the only source of albumin was donated blood. However, because of the inherent contamination risk and difficulty in de-contaminating the protein, efforts are now being made to produce albumin from genetically modified rice through isoelectric point manipulation.

Non-genetically modified Certified Organic hemp contains albumin in abundance (35% of the total protein). Potential applications for the use of hemp derived albumin include emergency medical treatment to stabilize blood pressure, as well as increased bioavailability of drugs and nutraceuticals that the body can normally not absorb. These facts are not widely known in the Hemp or dietary supplement industries. A source of non-genetically modified albumin could also be advantageous for marketing purposes.

Recent studies have demonstrated albumin's ability to enhance the water solubility of other molecules. Albumin plays the role of an “in-vivo solubilizing agent” allowing the solubilization of a wide range of biomolecules and drugs in a hydrophilic medium, i.e. the plasma. The solubility enhancement properties of albumin are mainly due to its ability to form reversible binding complexes with ligands. This allows the bound molecule to flow in the blood at concentrations higher than that of its initial solubility. Albumin has two main sites that bind the ligands mainly by hydrophobic and electrostatic interactions. Although the overall charge of albumin is negative at the physiological pH, the two principal binding sites are positively charged which promotes the binding of anionic molecules. Furthermore, albumin has several secondary binding sites increasing the number of bound molecules, e.g. up to seven fatty acid molecules. Amongst substances showing the highest affinity to albumin, anionic molecules (weak acid) and hydrophobic molecules of medium size (100-600 Da); poorly soluble drugs. Additionally, albumin molecules possess numerous accessible free amino and carboxyl groups amenable to forming highly soluble salts with acidic or basic drugs, respectively.

### SUMMARY

Embodiments of the present disclosure provide for the use of albumin derived from any source, including but not limited to any plant-based, human, or animal sources, as well as synthetic sources such as yeast or bacterial fermentation, to be used as an emulsifier to assist in blending otherwise immiscible components.

Some embodiments also provide for the use of albumin derived from any source, including but not limited to any plant-based, human, or animal sources, as well as synthetic sources such as yeast or bacterial fermentation, to be used as a drug carrier.

Additional embodiments provide for the use of albumin derived from any source, including but not limited to any plant-based, human, or animal sources, as well as synthetic sources such as yeast or bacterial fermentation, to be used to increase the bioavailability of drugs or dietary compounds that have poor water solubility.

In some embodiments, a method is provided for blending immiscible components using an emulsifying agent, comprising the step of using protein as the emulsifying agent. The source of protein can be albumin from any source, wherein blending results in an emulsion. The protein fraction can be used in a dry, gelatinous or aqueous form and may be stabilized by the addition of a flow agent. The protein fraction can be wet milled, resulting in a protein larger than 5 kDa, a concentration that varies from 40 to 99%, with solubility in the pH range of 8.0 to 12.0. Further, the emulsion may have a loading capacity up to 60%. The stability of the emulsion may be extended with the addition of nano-cellulose and the particle size is approximately 50 nm.

In some embodiments, a method is provided for producing micro- and nano-encapsulations, comprising the steps of dissolving albumin in water, adding a lipid-based ingredient and mixing by sonication or high pressure homogenisation. The encapsulated product may be soluble in oil or water and may contain a surfactant comprised of water soluble protein. The encapsulated product may be coated by a water soluble, plant-based protein and may be used as a carrier for drugs or nutraceuticals. The encapsulated product may also be used to increase the bioavailability of drugs. In some embodiments, the albumin may be obtained by using polymers derived by algae. The albumin may be concentrated by isoelectric-point manipulation, water-salt dialysis, or ultrafiltration. In some embodiments, the albumin may be enzymatically or chemically hydrolyzed. The albumin may be treated with absorbents or chemicals to remove color and flavour. Additional embodiments comprise a method where the lipid-based component is selected from the group consisting of cannabinoid oils, edible oils, pharmaceutical lipids and combination thereof, wherein the lipid-based component remains liquid at ambient temperature.

In additional embodiments, a method is provided for using albumin as a drug carrier, comprising the step of binding albumin to a drug, and orally delivering the drug carrier to a mammal. The drug may be ingestible or injectable. The drug may be a cannabinoid.

In some embodiments, a method is provided for improving water solubility of compounds by adding emulsified albumin to a compound, where the compound is a dietary compound. The dietary compound may have a lipid-based component.

### BRIEF DESCRIPTION OF THE FIGURES

FIG. 1 shows albumin protein (shown by light colored spheres) carrying fatty acid molecules (shown by darker colored spheres, also indicated by arrows).

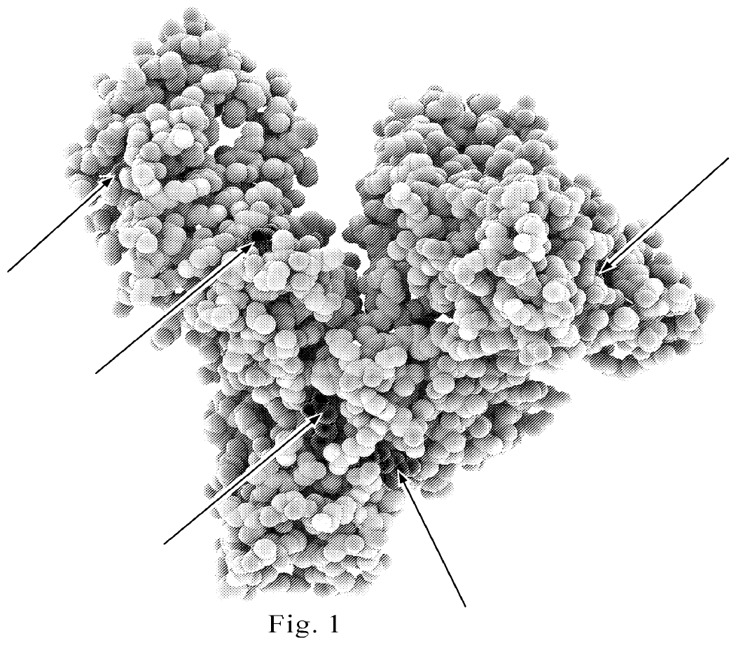

FIG. 2 is a flowchart that illustrates the process of separating and extracting hemp protein from hemp seeds. The source material (hemp seeds) can be replaced with a myriad of plant and/or animal material. The isolated protein will have a molecular weight above 5 kDa.

### DETAILED DESCRIPTION

Reference will now be made in detail to representative embodiments of the disclosure. While the disclosure describes multiple embodiments, it will be understood that there is no intent to be limited to those embodiments. On the contrary, the disclosure is intended to cover all alternatives, modifications, and equivalents that may be included as defined by the claims.

One skilled in the art will recognize many methods and materials similar or equivalent to those described herein, which could be used in and are within the scope of the practice of the present disclosure and is in no way limited to the methods and materials described.

All publications, published patent documents, and patent applications cited in this application are indicative of the level of skill in the art(s) to which the application pertains. All publications, published patent documents, and patent applications cited herein are hereby incorporated by reference to the same extent as though each individual publication, published patent document, or patent application was specifically and individually indicated as being incorporated by reference.

In some embodiments, there is provided a method for microencapsulation and nanoencapsulation of products, wherein the emulsified product contains:
an active ingredient that is readily soluble in oil, and a surfactant comprised of protein (animal, fungi, whole plants, spent/process material, foliage/seed) from polymeric separation, that is soluble in water.

In some embodiments, the source material with higher concentration of proteins are more favorable such as hemp seeds, almonds, or chia seeds.

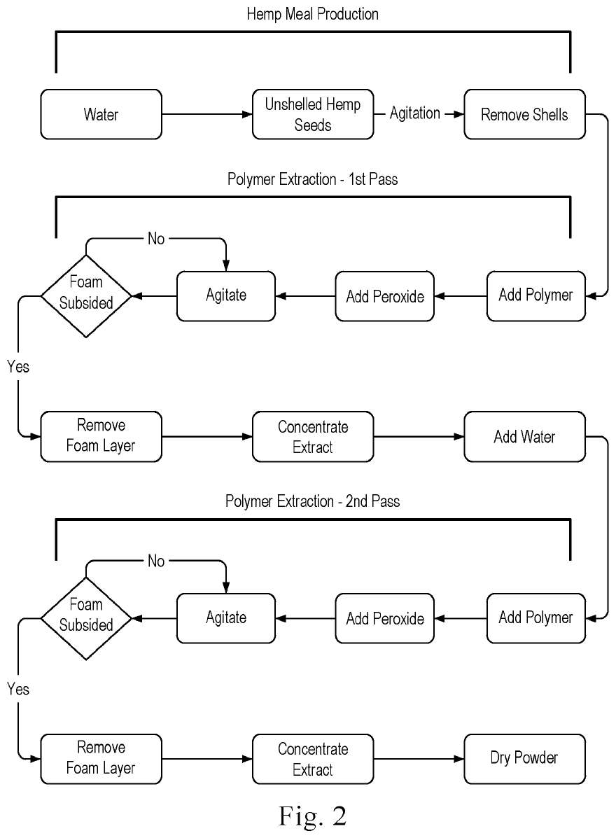

In some embodiments, the water-soluble protein fractions shall maintain solubility in a wide pH range of 8 to 12. In some embodiments, the water-soluble protein fractions shall maintain solubility in a wide pH range of 9 to 12. In some embodiments, the water-soluble protein fractions shall maintain solubility in a wide pH range of 10 to 12. In some embodiments, the isolated proteins will be larger than 5 kDa. In some embodiments, the isolated proteins will be larger than 4 kDa. In some embodiments, the isolated proteins will be larger than 4.5 kDa. In some embodiments, the isolated proteins will be larger than 5.5 kDa. In some embodiments, the isolated proteins will be larger than 6 kDa. Separation of the desired proteins from source material is accomplished using polymers derived from algae (described in US 2020/0231928 A1). Concentration of the separated protein can be achieved through isoelectric point manipulation, water-salt dialysis, or ultrafiltration. An example of the method is shown in FIG. 2 where the unshelled hemp seeds in FIG. 2 can be replaced with any material listed above as surfactant starting material.

In some embodiments, the isolated protein fraction can be used as an aqueous solution, a gelatinous suspension, or dried. The effective protein concentration may vary from 40 to 99% (w/w); this range includes a mixture of proteins, including the protein of interest (albumin). In one embodiment, the effective protein concentration may vary from 50 to 99% (w/w); this range includes a mixture of proteins, including the protein of interest (albumin). In one embodiment, the effective protein concentration may vary from 60 to 99% (w/w); this range includes a mixture of proteins, including the protein of interest (albumin). In one embodiment the effective protein concentration may vary from 70 to 99% (w/w); this range includes a mixture of proteins, including the protein of interest (albumin). In one embodiment, the effective protein concentration may vary from 40 to 50% (w/w); this range includes a mixture of proteins, including the protein of interest (albumin).

The isolated protein fraction may also be treated with absorbents or chemicals to remove color and flavour. The structure of the protein can be changed by hydrolysis. Hydrolysis can be accomplished via enzymes such as pancreatin, pepsin, papain, ficin, bromelain, alcalase, and/or chemicals (e.g. pH modulation).

Protein fractions have shown applicability towards encapsulating (nano and micro) lipid based components. Lipid components may include cannabinoid oils, pharmaceuticals and edible oils (Omegas). The encapsulated oil may be crude, filtered, distilled or refined as long as the material remains liquid at ambient temperatures.

In one embodiment, stable emulsions have a lipid load greater than 20%. In one embodiment, stable emulsions have a lipid load greater than 10%. In one embodiment, stable emulsions have a lipid load greater than 30%. In one embodiment, stable emulsions have a lipid load greater than 40%.

Encapsulated products are made by dissolving the concentrated protein fraction in water in the first container, and lipid based ingredients are combined in a second container. The contents in the first container are slowly added to the second container with constant agitation. In one embodiment, effective agitation is accomplished with sonication (20 kHz), or (10-30 kHz) or high pressure homogenization (25,000 to 40,000 psi) or high pressure homogenization (30,000 to 40,000 psi) or high pressure homogenization (20,000 to 40,000 psi).

In some embodiments, such as shown in FIG. 2, proteins included in the albumin protein production are isolated by agitation in the presence of algal polymers and peroxide.

In some embodiments, liquid emulsions can be stabilized by adding additional oils or thickening agents to match lipid phase and aqueous phase Hydrophobic Lipophilic Balance (HLB) and viscosities. Powdered encapsulations can be stabilized with the addition of flow agents such as dextrin or silicon dioxide.

In some embodiments, the separation process is followed by forming a stable emulsion of the protein with a lipid based active component.

In some embodiments, the stable emulsion consisting of a microencapsulated lipid is dried via spray dryer or evaporative plate drying.

In some embodiments, the stable emulsion consisting of a microencapsulated lipid is incorporated into a gelatinous matrix.

### Definitions

Unless defined otherwise, technical and scientific terms used herein have the same meaning as commonly understood by one of ordinary skill in the art to which this disclosure belongs. Although any methods, devices, and materials similar or equivalent to those described herein can be used in the practice or testing of the method, the preferred methods, devices, and materials are now described.

As used in this application, including the appended claims, the singular forms “a,” “an,” and “the” include plural references, unless the content clearly dictates otherwise, and are used interchangeably with “at least one” and “one or more.” Thus, reference to “a protein” includes a plurality of proteins, and the like.

As used herein, the term “about” represents an insignificant modification or variation of the numerical value such that the basic function of the item to which the numerical value relates is unchanged.

As used herein, the terms “comprises,” “comprising,” “includes,” “including,” “contains,” “containing,” and any variations thereof, are intended to cover a non-exclusive inclusion, such that a process, method, product-by-process, or composition of matter that comprises, includes, or contains an element or list of elements does not include only those elements but may include other elements not expressly listed or inherent to such process, method, product-by-process, or composition of matter.

As used herein, the term “homogeniser” refers to an agent or device assisting the process of converting immiscible liquids into an emulsion.

As used herein, the term “emulsifier” refers to an agent that assists in blending immiscible liquids into a stable solution. In general, emulsions with small particles tend to be more stable.

As used herein, the term “bioavailability” refers to the degree and rate at which a substance is absorbed into a living system or is made available at the site of physiological activity.

As used herein, the term “absorption” refers to the transfer of substances from the blood, into cells, tissues, or organs, to be supplied to the rest of the body.

As used herein, the term “carrier” refers to a substrate used to deliver a substance which in turn serves to improve the selectivity, effectiveness, and/or safety of administration of the substance to a patent in need.

As used herein, the term “loading capacity” refers to the amount of oil that can be emulsified when mixed with albumin, as a weight/weight percent.

As used herein, the term “flow agent” refers to a substance used to improve physical properties of the product, such as dextrin or silicon dioxide and the like.

As used herein, the term “absorbent” refers to a substance, such as carbon, activated charcoal and the like.

As used herein, the term “nutraceuticals” refers to any product derived from food sources, that contains health-giving additives and having medicinal benefit, such as ubiquinone, s-adenosylmethionine, glucosamine and the like.

As used herein, the term “drug” refers to any chemical substance that causes a change in an organism's physiology or psychology. The term “drug” includes, but is not limited to, THC, cannabinoids, NSAIDs, nicotine, antipsychotics, antiemetics, statins, etc.

As used herein, the phrase “foaming agent” refers to a surfactant, which when present in small amounts, facilitates the formation of a foam, or enhances its stability by inhibiting the coalescence of bubbles. Foaming agents can be inorganic chemicals such as sodium bicarbonate, ammonium carbonate, ammonium bicarbonate, and calcium azide and the like, as well as organic foaming agents such as azodicarbonamide, benzenesulfonyl hydrazide and dinitrosopentamethylene tetramine, and the like.

As used herein, the phrase “isoelectric point manipulation” refers to isolation of a protein by precipitation at a pH where the protein has zero net charge.

As used herein, the phrase “stability of the emulsion” refers to the emulsion ability to prevent coagulation, flocculation, sedimentation or phenomena akin to Ostwald ripening. The stability may be impacted by pH or temperature modulation.

### Processing Steps

1. Purified hemp albumin protein was wet milled using a high pressure homogeniser; resulting average particle size was measured as 50 nm (nano meters).

2. At a concentration of 1%, the resultant protein product was tested as an emulsifier and found that it could emulsify vegetable oils with very high loading capacity. Up to 60% oil could remain in emulsion using 1% hemp albumin.

3. The protein product was tested as an emulsifier for cannabinoids and found that it created highly stable emulsions with small particle size, less than 100 nm. It was found that addition of small quantities (e.g., 0.5%) of crystalline nano-cellulose further extended stability of the emulsion to greater than 24 months with no emulsion separation.

4. Albumin cannabinoid emulsion was further tested in bioavailability studies. It was found that cannabinoid absorption is enhanced when emulsified with albumin protein. Specifically, bioavailability was increased by a factor of 20 resulting in near 100% absorption of ingested cannabinoids within a 6 hour period.

5. Safety trials revealed that hemp albumin can be injected into the human blood circulation with negligible adverse effects, thus pure hemp albumin is suitable to be used as a pharmaceutical drug carrier for both ingestible and injectable drugs.

6. Hemp albumin dispersed in water at concentrations ranging from 5 to 20%, and more specifically 10%, can form a clear gel when heated for 15 minutes at temperatures between 6° and 90° C., more specifically 85° C., followed by homogenisation with the addition of a small amount of salt, typically 2% of the total mixture. This gel has application in food, Pharma and cosmetics.

### Example 1

1. Use of hemp derived albumin as plant based emulsifier:
a. Traditional saponin-type emulsifiers, such as Quillaja, are limited by their emulsion particle size (250 nm) during reasonable processing. Substituting albumin (hemp) will allow the end user to reach particle sizes of approximately 50 nm using a high pressure homogenizer. This technology and ingredient can be paired with bioactive constituents, resulting in increased biological uptake and thus efficacy.

### Example 2

1. Use of hemp derived albumin as plant based amino acids for Agriculture:

a. Amino acids chelate minerals (make them bioavailable), they bolster the immune system of the plant, stimulate plant growth and enhance the quality of fruit and vegetables. Currently, amino acids used in agriculture are derived from fish which is not sustainable.

### Example 3

1 Use of hemp derived albumin for emergency medical treatment:

a. Albumin is the most abundant protein in circulating blood plasma. It represents half to the total protein content of plasma in healthy humans which is about 5% of the plasma. In an 154 pound (70 kg) adult there will be 140 g of Albumin. Albumin exerts osmotic pressure which keeps water in the blood, maintaining blood pressure. If blood is lost, administering Albumin is used to maintain blood pressure and keep the patient alive. There is demand for a clean source of Albumin for emergency medical care. Donated blood is very difficult to keep free from contamination. Currently genetically modified rice is being used as a source of Albumin. Hemp could easily be a vastly superior source of this essential protein.

### Example 4

1. Use of hemp derived albumin as a drug carrier:

a. When drugs and other bioactive molecules enter the blood they are attached to an Albumin molecule to keep them water soluble for transport through the body. Examples include hormones, fatty acids and cannabinoids. Albumin is an ideal carrier for administering drugs and other therapeutic agents to the body by using Albumin to create nano emulsions that are then introduced to the body intravenously or orally.

### Example 5

1 Use of hemp derived albumin as a source of bioactive peptides:

a. Hydrolysing Albumin with enzymes or microbes yields protein fragments called peptides. These peptides are used for medical uses such as reversing high blood pressure and dementia.

1. A method for blending immiscible components comprising blending with an emulsifying agent, wherein the emulsifying agent comprises protein fraction.

2. The method according to claim 1 wherein blending comprises a wet milled protein fraction and a high-pressure homogeniser; wherein blending results in formation of an emulsion.

3. The method according to claim 2, wherein the protein fraction is albumin.

4. The method according to claim 3, wherein the source of albumin is selected from the group consisting of hemp seed, a plant, an animal, a yeast or a bacteria.

5-6. (canceled)

7. The method according to claim 1, wherein the protein fraction can be used in a form selected from the group consisting of a dried form, a gelatinous form and an aqueous form.

8-9. (canceled)

10. The method according to claim 7, wherein the protein fraction is stabilized by the addition of flow agents.

11. The method according to claim 1, wherein the protein concentration varies from 40 to 99%, and wherein the protein fractions are soluble at a pH range of 8.0-12.0.

12-15. (canceled)

16. The method of claim 2, wherein stability of the emulsion produced may be extended with the addition of nano-cellulose.

17. (canceled)

18. A method for producing micro- and nano-encapsulations, comprising the steps of dissolving albumin in water, adding a lipid-based ingredient and mixing by sonication or high pressure homogenization, wherein the lipid-based component remains liquid at ambient temperature.

19. The method of claim 18, wherein the encapsulated product is soluble in a liquid selected from the group consisting of oil and water.

20. (canceled)

21. The method of claim 18, wherein the encapsulated product contains a surfactant comprised of water-soluble protein, wherein the encapsulated product may be coated with a water soluble, plant-based protein.

22. (canceled)

23. The method of claim 18, wherein the encapsulated product is used as a carrier selected from the group consisting of a drug carrier and a nutraceutical carrier, wherein the encapsulated product is used to increase the bioavailability of drugs.

24-25. (canceled)

26. The method of claim 18, wherein the albumin is obtained using polymers derived from algae.

27. The method of claim 18, wherein the albumin is concentrated by a process selected from the group consisting of isoelectric point manipulation, water-salt dialysis, and ultrafiltration.

28-29. (canceled)

30. The method of claim 18, wherein the albumin is hydrolyzed by a process selected from the group consisting of enzymatic hydrolyzation and chemical hydrolyzation.

31-32. (canceled)

33. The method of claim 18, wherein the lipid-based component is selected from the group consisting of cannabinoid oils, edible oils and pharmaceutical lipids.

34. (canceled)

35. A method for using albumin as a drug carrier, comprising the steps of binding albumin to a drug, and orally delivering the drug carrier to a mammal.

36. The method of claim 35, wherein the drug is ingestible or injectable.

37. (canceled)

38. The method of claim 35 wherein the drug is a cannabinoid.

39. A method of improving water solubility of compounds, comprising the steps of adding emulsified albumin to a dietary compound.

40-41. (canceled)
